(19) United States
(12) Patent Application Publication
(10) Pub. No.: US 2024/0399076 A1
(43) Pub. Date: Dec. 5, 2024

(54) Device to Control the Internal Body Pressure During Use of a Medical-Technical Pump

(71) Applicant: W.O.M. World of Medicine GmbH, Berlin (DE)

(72) Inventor: Andreas Zeyssig, Berlin (DE)

(73) Assignee: W.O.M World of Medicine GmbH, Berlin (DE)

(21) Appl. No.: 18/797,196

(22) Filed: Aug. 7, 2024

Related U.S. Application Data
(63) Continuation of application No. 14/865,520, filed on Sep. 25, 2015.

(30) Foreign Application Priority Data
Mar. 28, 2014 (DE) 102014004480.7

(51) Int. Cl. A61M 13/00; A61B 17/00; A61B 17/34

(52) U.S. Cl. CPC A61M 13/003; A61B 17/00; A61B 17/3474; A61M 2205/3331; A61M 2205/3334; A61M 2205/3337; A61M 2205/3344; A61M 2205/3355; A61M 2205/50; A61M 2205/52; A61M 2205/75; A61M 2210/086

(57) ABSTRACT

The present invention relates to a method for controlling the internal body pressure, e.g., the joint pressure using a medical-technical fluid pump, and to a device for carrying out said method.

### CROSS-REFERENCE TO RELATED APPLICATIONS

This application is a continuation of U.S. Patent Application Ser. No. 14/865,520, filed on Sep. 25, 2015, entitled Device to control the internal body pressure during use of a medical-technical pump, which is a national continuation-in-part application based on PCT International Application No. PCT/DE2015/000152, filed on Mar. 30, 2015, entitled Method and device for regulating the body's internal pressure during use of a medical pump, which claims the benefit of priority to German Patent Application No. DE 10 2014 004480.7 filed on Mar. 28, 2014, the entire contents of each of these applications are hereby incorporated by reference.

### BACKGROUND OF THE INVENTION

The present invention relates to a method for controlling the internal body pressure, e.g., the joint pressure using a medical-technical fluid pump, and to a device for carrying out said method.

During various medical interventions in the body interior, fluids, e.g., gases or liquids are introduced into the body interior and are removed therefrom. An example is arthroscopy, where, for instance for the purpose of a knee joint examination or a therapeutic treatment, the knee is rinsed with a rinsing liquid. Another exemplary treatment is laparoscopy, where during a therapeutic intervention, gases (e.g., CO₂) are supplied to the body interior. For these procedures, measuring, controlling, and in particular limiting the pressure in the body interior is very important. During therapeutic interventions, it is in particular necessary, to guarantee a certain fluid flow, in order to rinse off for instance smoke or blood from the body interior, simultaneously however to limit the pressure, in order to not unnecessarily damage the body tissue. For this purpose, various solutions can be found in prior art. A simple solution of the problem is to introduce a pressure sensor immediately into the body interior. The disadvantage of this solution is, inter alia, that additional space in the body interior is required, which is not available in particular for small body cavities (e.g., for arthroscopy). Furthermore, every additional duct into the body interior will increase the risk of infection being always present.

Another solution is to measure the pressure in the supply line. This pressure, however, differs more or less from the actual pressure in the body interior, due to the flow-dynamic conditions in the supply and discharge lines. Since this difference of the measured pressure from the actual value depends in a non-linear way on a series of parameters (e.g., flow speed, length of lines, diameter of lines, etc.), a simple correction is not possible.

Given this background, it is the object to provide a medical device carrying out an improved method for measuring, controlling, and in particular for limiting the pressure, which overcomes the above disadvantages. For achieving this object, the device according to claim 1 is proposed. Advantageous embodiments are subject matter of the dependent claims.

### SUMMARY OF THE INVENTION

The method according to the invention is substantially based on that for measuring and controlling the internal body pressure, a mathematical model of the control section is used. For this purpose, the complete system consisting of pump motor, supply line, pressure sensor, medical insertion device (e.g., trocar with optical system), body cavity and fluid outlet (e.g., suction device) are described by a set of differential equations and summarized in a so-called state space model. Provided that the parameters of the model have been determined sufficiently precisely, there follows, with the same input variable, an estimation for the sensor pressure and the pressure in the body interior. By a comparison between the actual pressure and the estimated sensor pressure, deviations (so-called observer errors) can be detected. These may be caused, for instance, by different initial states (e.g., at the beginning of the operation, there is no information about the internal body pressure) or by a noisy measurement signal of the pressure sensor. If the observer error is fed back by using a feedback gain to the input of the state space module, the error will decay, and as a result, a precise estimation for the pressure in the body interior is obtained. The advantage of the proposed method is, inter alia, that for the measurement of the internal body pressure, no additional pressure sensor is required. As a result, a distinctly better measurement of the internal body pressure is obtained, even under modification of the influencing variables, such as, e.g., the discharge of liquids or when switching a suction pump on, or the like.

Preferably, the method according to the invention is configured such that the estimation system is realized as a state observer. Such state observers are explained, e.g., in textbooks of control engineering. Depending on the actual problem, weighting matrices can be taken into account in the state observer, which represent a possible system and measurement noise. This can be caused, on the one hand, by stochastically acting process interferences or by deficiencies of the sensor system. Thereby, the result of the estimation of the state variable to be reconstructed can be improved. The type of state observer may be configured in a continuous or discrete-time manner, and this applies for linear and non-linear systems. Examples of state observers include: linear and non-linear Luenberger observer for deterministic system behavior, linear and non-linear Kalman filter for deterministic and stochastic system behavior, high-gain observer for deterministic systems (feedback gain is set such that the linear portion of the system dominates), etc.

A particular embodiment of a device realizing the above method is a peristaltic hose pump, as it is used, for instance, in arthroscopy. It contains a controllable motor supplying the peristaltic pump power. Via a supply line (e.g., hose and a trocar with optical system) the pumped liquid is conducted into the body interior (e.g., the knee joint). From the knee joint, the liquid can be discharged in different ways. Either via a separate skin incision or via additional discharge lines for draining and shaver. The latter are often connected to a suction or vacuum pump. By the method according to the invention, the actual pressure in the joint is estimated using the measurement data of the pressure sensor and is controlled by the pump motor.

An alternative embodiment of the invention provides an insufflator, as it is used for laparoscopy. By means of the insufflator, a gas is conducted into the body interior (e.g., the abdomen). Gas discharge from the body interior is realized, here too, for instance, by an extraction line. Here too, a pressure measurement is made in the supply line. The actual pressure in the body interior is estimated by the method according to the invention and serves for controlling the insufflator. As a result, a device is obtained that ensures a precise measurement and control of the pressure even under extreme conditions, e.g., when switching a suction device on and off.

### BRIEF DESCRIPTION OF THE DRAWINGS

FIG. 1 shows a direct fault-free pressure measurement in the joint, a pump is controlled, which ensures the liquid flow into the joint.

FIG. 2 shows the actual pressure in the joint is estimated based on the input speed of the pump motor and the data of the pressure sensor in the hose using a state observer.

FIG. 3 shows a flowchart of the software module.

FIG. 4A shows the measured sensor pressure is compared to the estimate sensor pressure.

FIG. 4B shows the measured joint pressure is compared to the estimated joint pressure.

FIG. 4C shows the liquid discharge estimated by the Kalman filter.

FIG. 5 shows the results of comparative measurements of a classic peristaltic pump with a pressure sensor in the supply line (designated A114) and a pump according to the invention with a mathematical estimation system, which, as am input variable, receives the pressure of a pressure sensor as measured in the supply line (designated A124).

### DETAILED DESCRIPTION OF THE INVENTION

Embodiments of the invention are illustrated in the drawings and are explained in more detail in the following.

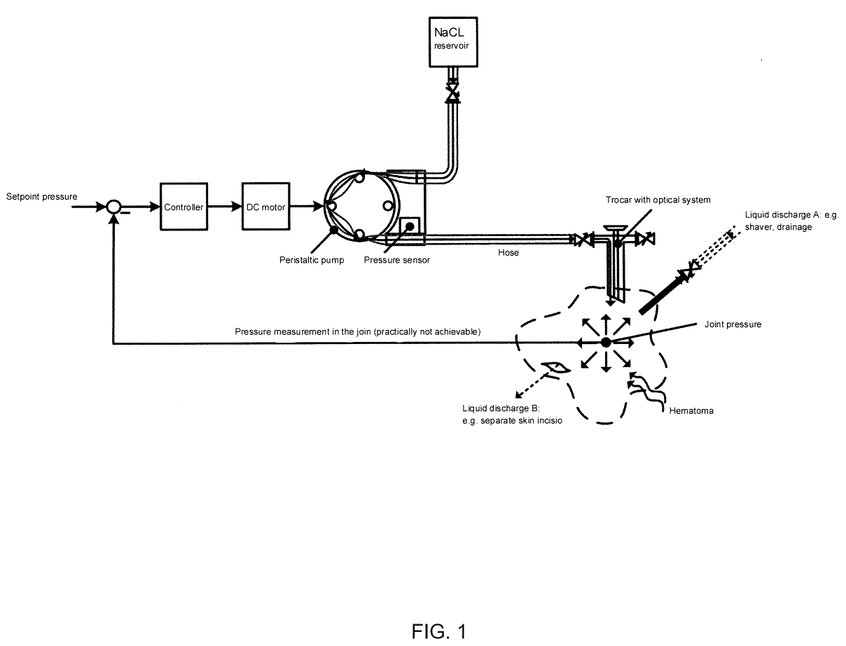

FIG. 1 shows schematically the ideal situation for therapists: By a direct fault-free pressure measurement in the joint, a pump is controlled, which ensures the liquid flow into the joint. Since such a direct pressure measurement is practically not possible, FIG. 1 shows just the intended aim.

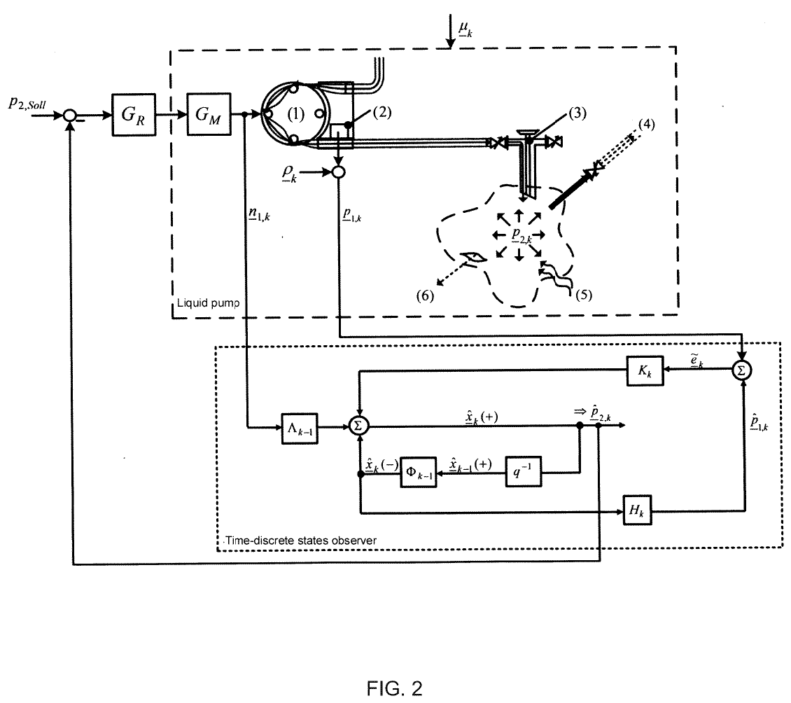

FIG. 2 shows an example of a solution according to the invention: the actual pressure in the joint is estimated based on the input speed of the pump motor and the data of the pressure sensor in the hose using a state observer. The detected deviations of the measured pressure from the estimated pressure in the hose are fed back via a feedback vector to the input of the state space model. Furthermore, FIG. 2 shows the state equations used for this model in the state space as a block diagram. For the sake of simplicity, the representation is shown for a time-discrete linear system, which may be a special case of the non-linear description form. An explicit notation for marking the deviations about a possible operating point is dispensed with in the following. Furthermore, it is assumed that the system to be observed is disturbed by to zero-mean, normally distributed, uncorrelated, white noise processes. This is taken into account when calculating the feedback vector.

The numbers shown in the figure have the following meaning:

| | |
|---|---|
| (1) | Peristaltic pump |
| (2) | Pressure sensor in hose |
| (3) | Trocar-optical system-combination |
| (4) | Liquid discharge via, e.g., drainage and shaver |
| (5) | Hematoma |
| (6) | Liquid discharge via a separate skin incision |
| G_{R} | Pressure controller |
| G_{M} | Speed-controlled DC motor |
| p_{2, Soll} | Setpoint joint pressure |
| μₖ | Zero-mean, normally distributed, |
| | uncorrelated process noise |
| ρₖ | Zero-mean, normally distributed, |
| | uncorrelated measurement noise |
| n_{1, k} | Measurable input speed at time tₖ |
| p_{1, k} | Measurable pressure in hose at time tₖ |
| p_{2, k} | Non-measurable pressure in control region at time tₖ |
| [symbol] | Estimated pressure in hose at time tₖ |
| [symbol] | Estimated pressure in control region at time tₖ |
| Λₖ₋₁ | Time-variant control matrix of discrete-time system |
| Φₖ₋₁ | Time-variant transition matrix of discrete-time system |
| Hₖ | Discrete-time output matrix |
| Kₖ | Feedback vector |
| [symbol] | Observer error |
| [symbol] (+) | Updated state estimation after |
| | measurement trim at time tₖ |
| [symbol] (+) | Updated state estimation after |
| | measurement trim at time tₖ₋₁ |
| [symbol] (−) | Extrapolated state estimation after |
| | measurement trim at time tₖ |
| q⁻¹ | Backward shift operator |

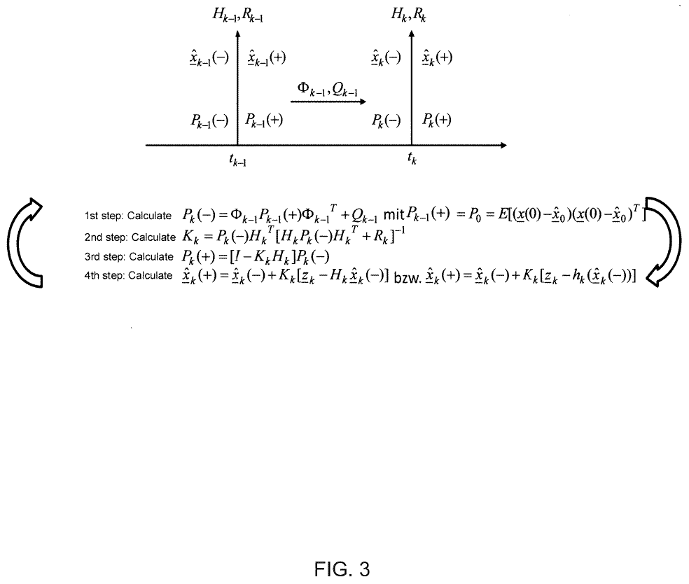

The practical implementation of the above method suitably occurs on a microcontroller that is part of the medical-technical device. It is provided in a conventional way with inputs and outputs and memories. The mathematical operations are performed in the form of a software module. A flowchart of the software module is shown in FIG. 3. The software may be stored on an own memory chip, for instance, an EPROM.

The variables in FIG. 3 not yet known are as follows:

| | |
|---|---|
| Rₖ | Covariance matrix of measurement noise |
| Qₖ₋₁ | Covariance matrix of process noise |
| Pₖ (−) | Extrapolated error covariance matrix |
| Pₖ₋₁ (+) | Updated error covariance matrix after a measurement |

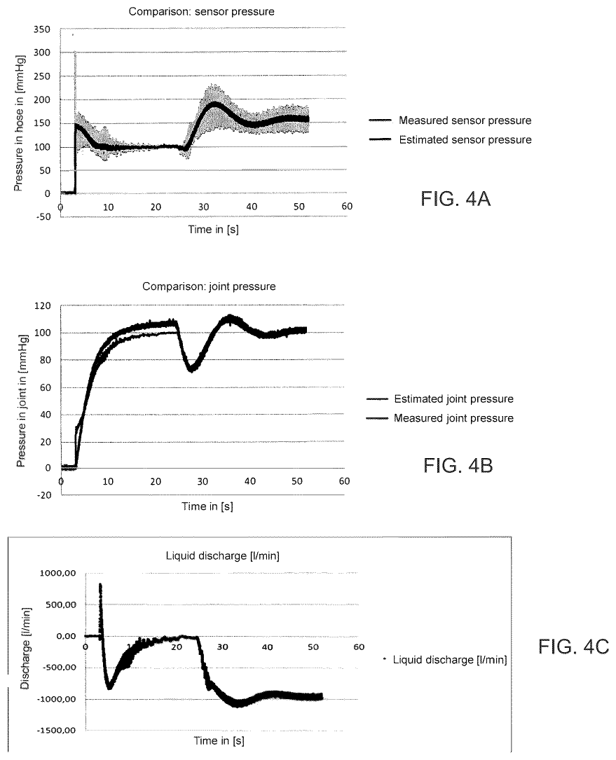

FIG. 4 shows the obtained results. In FIG. 4A, the measured sensor pressure is compared to the estimated sensor pressure, and in FIG. 4B, the measured joint pressure is compared to the estimated joint pressure. The graphs shown in FIG. 4 indicate a control process, wherein as a control variable, the joint pressure reconstructed by the state observer is used. Using FIG. 4B, it becomes clear that the estimated joint pressure is nearly identical to the measured joint pressure. In FIG. 4C, the liquid discharge estimated by the Kalman filter is shown.

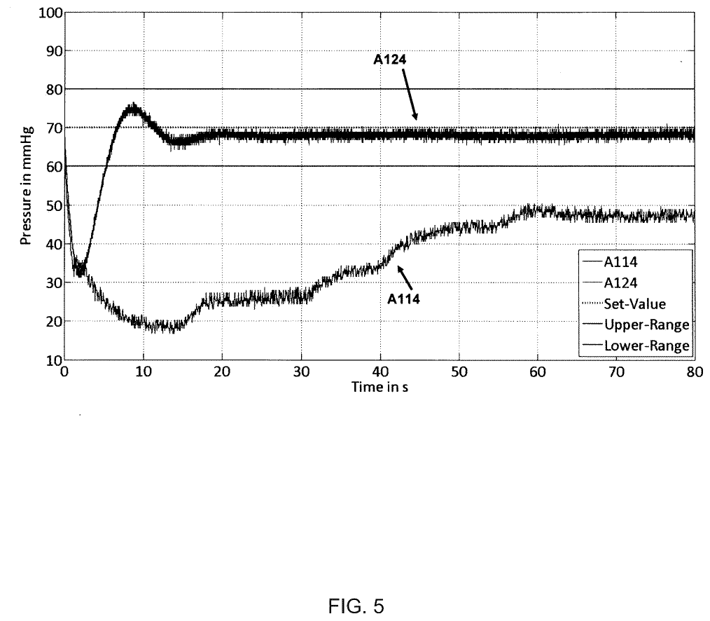

FIG. 5 shows the results of comparative measurements. In one experiment, two medical pumps were compared. That is, on the one hand, a classic peristaltic pump with a pressure sensor in the supply line (designated A114) and a pump according to the invention with a mathematical estimation system, which, as an input variable, receives the pressure of a pressure sensor as measured in the supply line (designated A124). Both pumps were connected to a dummy, which simulates the conditions in an actual body cavity. Shown are the values actually measured within the dummy by a separate sensor. At both pumps, a setpoint pressure of 70 mmHg was adjusted (designated “set value”), wherein pressure variations of ±10 mmHg were considered acceptable (designated “upper range” or “lower range”, respectively).

The comparison of the curves shows that the pump designed according to the invention reaches after five seconds already a pressure within the mentioned range of 60 to 80 mmHg and very precisely stays at the intended pressure after a short attack time. In contrast, in the prior art pump, only after 60 seconds a reasonably stable value is achieved, however outside the adjusted range. Even if here a recalibration would be performed, there remain clear deviations from the set value within the first 60 seconds and the larger variations in the course of time.

Therefore, the comparison of the two pumps clearly shows the surprising advantage of the system according to the invention, namely that the pressure set value is reached markedly faster, and can further be kept constant over time in a much better way. Such an improvement cannot be taken from prior art.

The man skilled in the art can, based on the above description, in particular the descriptions in FIG. 2 and the professional literature well-known at the time of the application, implement further embodiments of the invention, without any inventiveness being required.

What is claimed is:

1. A medical device for introducing fluids into a body cavity during a medical procedure, comprising:
a controllable fluid pump,
a supply line operatively connected to the fluid pump for supplying a fluid into the body cavity,
a pressure sensor in the supply line,
a second line for enabling the fluid to flow out of the body cavity,
a controller for controlling the fluid pump,
a software module executed on a microcontroller, the software module being programmed as a state observer, namely a linear or non-linear Luenberger observer or
a linear or non-linear Kalman filter,
wherein a pressure measured by the pressure sensor is a measurable state variable for calculating an observer error for the state observer, the state observer being described by a state space model, which describes the mathematical-physical relation, which estimates an actual pressure in the body cavity and controls a pump speed of the fluid pump based on this estimated actual pressure so as to reach a selected setpoint pressure in less than 20 seconds and maintain such selected setpoint pressure in a range of +/−10 mmHg during use of the device during the medical procedure.

2. The medical device according to claim 1, wherein the fluid supplied to the body cavity is a gas.

3. The medical device according to claim 2, wherein the device is an insufflator.

4. The medical device according to claim 1, wherein the fluid supplied to the body cavity is a liquid.

5. The medical device according to claim 4, wherein the device is adapted for use in arthroscopy.

6. A medical device for introducing fluids into a body cavity during a medical procedure, comprising:
a controllable fluid pump,
a supply line operatively connected to the fluid pump for supplying a fluid into the body cavity,
a pressure sensor in the supply line,
a second line for enabling the fluid to be discharged from the body cavity,
a controller for controlling the fluid pump,
a software module executed on a microcontroller, the software module being programmed as a time-discrete state observer,
wherein a pressure measured by the pressure sensor and an input speed of the fluid pump are measurable state variables for calculating an observer error for the state observer, the state observer being a state space model which estimates an actual pressure in the body cavity and controls a pump speed of the fluid pump based on this estimated actual pressure so as to reach a desired setpoint pressure.

7. The medical device according to claim 6, wherein the fluid supplied to the body cavity is a gas.

8. The medical device according to claim 7, wherein the device is an insufflator.

9. The medical device according to claim 6, wherein the fluid supplied to the body cavity is a liquid.

10. The medical device according to claim 9, wherein the device is adapted for use in arthroscopy.

11. The medical device according to claim 6, wherein the state observer is a linear or non- linear Luenberger observer or a linear or non-linear Kalman filter.

12. The medical device according to claim 11, wherein the amount of liquid discharged from the body cavity is estimated by the Kalman filter.
